(19) United States
(12) Patent Application Publication
(10) Pub. No.: US 2008/0300401 A1
(43) Pub. Date: Dec. 4, 2008

(54) Novel chlorination process for preparing sucralose

(75) Inventor: Shaojun Xu, Hangzhou (CN)

Correspondence Address: BUCHANAN, INGERSOLL & ROONEY PC, POST OFFICE BOX 1404, ALEXANDRIA, VA, 22313-1404, US

(73) Assignee: POLYMED THERAPEUTICS, INC., Houston, TX (US)

(21) Appl. No.: 12/003,850

(22) Filed: Jan. 2, 2008

Related U.S. Application Data
(63) Continuation-in-part of application No. 11/806,810, filed on Jun. 4, 2007.

(51) Int. Cl. C07H 1/06; C07H 1/00

(52) U.S. Cl. 536/127; 536/124

(57) ABSTRACT

A process for preparing a sucralose-6-ester, a key intermediate to sucralose. The process contains (a) creating a heterogeneous mixture comprising a first phase comprising a sucrose-6-ester and a second phase comprising a chlorinating reagent; and (b) reacting the sucrose-6-ester with the chlorinating reagent, to prepare a sucralose-6-ester. In addition, processes for preparing sucralose are provided.

### CROSS-REFERENCE TO RELATED APPLICATIONS

This application is a continuation-in-part of application Ser. No. 11/806,810, filed Jun. 4, 2007, the contents of which are incorporated herein by reference in its entirety.

### BACKGROUND OF THE INVENTION

1. Field of the Invention

The present invention relates to processes for preparing sucralose. In particular, the present invention relates to processes for preparing sucralose-6-esters and 4,1′,6′-trichloro-4,1′,6′-trideoxy galactosucrose-penta-acetate.

2. Description of the Related Art

Sucralose is a high intensity artificial sweetener having a sweetness about 600 times that of sucrose. Sucralose has been used as a food sweetener in many countries since its discovery in the 1970s. The chemical name of sucralose is 1,6-dichloro-1,6-dideoxy-β-D-frucofuranosyl 4-chloro-4-deoxy-α-D-galactopyranoside or 4,1′,6′-trichloro-4,1′,6′-trideoxygalactosucrose (TGS). The chemical structure of sucralose is represented by the following formula (I):

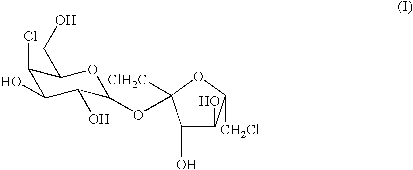

There are a number of processes for the preparation of sucralose, all of which involve selective chlorination of a sucrose molecule in the 4-,1′- and 6′-positions.

One of two main synthesis routes to sucralose involves the preparation of 2,3,6,3′,4′-penta-O-acetyl sucrose, in which the three hydroxyl groups to be chlorinated are unprotected, while all the remaining hydroxyl groups are protected in the form of acetates. 2,3,6,3′,4′-Penta-O-acetyl sucrose is then reacted with a chlorinating agent to afford 4,1′,6′-trichloro-4,1′,6′-trideoxygalactosucrose-penta-acetate (TOSPA), which in turn is hydrolyzed to produce sucralose. Selectively protecting the five positions of sucrose not to be chlorinated, while exposing the three positions to be chlorinated, imposes a number of technical difficulties. Moreover, this process involves multiple steps as well as complex operations and hence has little feasibility for large scale production of sucralose.

An alternative approach is to prepare a sucrose-6-ester which is then selectively chlorinated in the 4-,1′- and 6′-positions, followed by hydrolysis. This process is currently commonly used in the industry.

Selective chlorination of a compound containing multiple hydroxyl groups with different reactivity can be difficult. The hydroxyl groups at different positions of sucrose have different reactivity, which presents challenges for selective chlorination of sucrose. Thus, the main problem in the synthesis of TGS concerns selective chlorination, i.e., chlorination of the 4-,1′- and 6′-positions of a sucrose molecule without chlorination at other positions. Various studies show that the reactivity of the hydroxyl groups in sucrose towards chlorination is in the order of 6,6′>4>1′. These results are described, for example, by Fariclough et al., in Derivatives of β-D-Fructofuranosyl α-D-Galactopyranoside, Carbohydrate Research, 40, 285-298 (1975); and in GB 1,543,167 and GB 1,543,168. Therefore, when the 6-position of sucrose is protected, for example, in the form of an ester (i.e., sucrose-6-ester), it may be possible to selectively chlorinate the other desired hydroxyl groups, i.e., at the 4-, 1′- and 6′-positions, under appropriate conditions, to produce corresponding sucralose-6-ester.

Selective chlorination of sucrose and its derivatives may be effected by various methods. For example, Walter A. Szarek reported in Deoxyhalogeno Sugars, Advances in Carbohydrate Chemistry & Biochemistry 28, 225-306 (1973), the studies of various chlorinating agents for preparing chloro-deoxycarbohydrates. In addition, Viehe et al. reported in The Chemistry of Dichloromethyleneammonium Salts (“Phosgenimonium Salts”), Angew. Chem. Internat. Edit., 12(10), 806-18 (1973), the results of chlorination of hydroxyl groups using a Vilsmeier reagent formed from phosgene and N,N-dimethylformamide (DMF). Moreover, Benazza reported in Direct Regioselective Chlorination of Unprotected Hexitols and Pentitols by Vilsmeier and Haack's Salt, Tetrahedron Letter, 33 (34), 4901-4904 (1992), selective chlorination of hydroxyl groups. In all these studies, chlorination was performed in a homogeneous system, in which chlorination takes place with reasonable selectivity and yields.

U.S. Pat. No. 4,380,476 to Mufti et al. describes a process for the preparation of sucralose-6-acetate. In this process, a solution of sucrose-6-acetate in DMF is added to a Vilsmeier reagent in DMF. The reaction mixture is neutralized, and then concentrated under high vacuum (1 mmHg) and at a temperature of below 70° C. to remove DMF. The syrupy residue is preferably peracetylated by the treatment with acetic anhydride and pyridine at an elevated temperature, for example, 50° C., for 2 hours. The acetylation reaction is terminated with methanol, and concentrated under high vacuum to obtain a residue. The residue is then extracted with hot toluene (about 60° C.) and concentrated to produce a syrup. This syrup is dissolved in ethyl acetate, washed with water, dried and concentrated to obtain a second syrup. The second syrup is crystallized from ethanol two to three times to obtain TOSPA in 98% purity. TOSPA is deacetylated with sodium methoxide to prepare TGS.

U.S. Pat. No. 4,980,463 to Walkup et al. describes a similar process for the preparation of sucralose-6-esters. In this process, sucrose-6-benzoate or sucrose-6-acetate is dissolved in DMF and chlorinated with a Vilsmeier reagent. In this patent, a chlorinating reagent such as phosgene is added directly to a solution of a sucrose-6-ester in DMF, which is reversal of the addition sequence described in U.S. Pat. No. 4,380,476. According to Walkup et al., the reversed addition sequence improves reaction yields.

In both U.S. Pat. No. 4,380,476 and U.S. Pat. No. 4,980,463, the chlorination reactions were performed in a homogeneous reaction medium consisting essentially of DMF, and have certain drawbacks. For example, DMF has a relatively high boiling point (153° C.), which can render removal of DMF after the reaction by distillation very difficult. In addition, DMF is water miscible. Therefore, when DMF is used in large amounts, for example, as a reaction solvent, recovery of sucralose-6-esters from the reaction mixture by aqueous extraction can be complicated, because DMF and hence sucralose-6-esters tend to distribute in both aqueous and organic phases. Moreover, recycling of DMF recovered from the water-containing mixture during the workup process can be problematic. Furthermore, additional procedures may be necessary for the treatment of waste water prior to safe disposal. These result in a significant increase of the production costs. Additionally, the use of pyridine in the acetylation reaction also causes increases in the costs related to the production and waste treatment.

In U.S. Pat. No. 5,298,611 to Navia et al., sucralose-6-acetate is prepared in the same manner as described in U.S. Pat. No. 4,980,463. After the chlorination reaction is complete, the reaction mixture is neutralized with a base. The mixture containing sucralose-6-acetate, DMF, water, salts and chlorinated by-products is then subjected to a complex steam distillation process, thereby removing DMF from the mixture. After steam distillation, bottom products typically contain about 1-3 wt % sucralose-6-acetate, about 0.3-1.0 wt % of various other chlorodeoxysucrose derivatives, about 0.1-0.5 wt % of DMF, about 80-90 wt % of water and about 8-12 wt % of salts. Thereafter, the residue is concentrated to about half the volume and extracted with ethyl acetate. The organic phase of ethyl acetate is concentrated to produce a sucralose-6-acetate syrup, which in turn is reacted with acetic anhydride in the presence of pyridine at 50° C. for 24 hours, followed by addition of water at 0° C. to crystallize TOSPA. Although this process may provide improvements in recovery of DMF and acetylation conditions, it still does not resolve the problems related to recycling DMF.

There is interest in providing more efficient processes for preparing sucralose in a cost-effective manner.

### SUMMARY OF THE INVENTION

The present application provides a process for preparing a sucralose-6-ester, a key intermediate to sucralose, comprising:

(a) creating a heterogeneous mixture comprising a first phase comprising a sucrose-6-ester and a second phase comprising a chlorinating reagent; and

(b) reacting the sucrose-6-ester with the chlorinating reagent, to prepare a sucralose-6-ester.

The present application also provides a process for preparing sucralose comprising:

(a) creating a heterogeneous mixture comprising a first phase comprising a sucrose-6-ester and a second phase comprising a chlorinating reagent;

(b) reacting the sucrose-6-ester with the chlorinating reagent, to prepare a sucralose-6-ester; and

(c) deesterifying the sucralose-6-ester, to prepare sucralose.

### DETAILED DESCRIPTION OF THE PREFERRED EMBODIMENTS

As used herein, the term “heterogeneous system” is understood by those skilled in the art and generally refers to a non-uniform composition containing two or more phases, e.g., liquid/liquid, liquid/solid, liquid/liquid/liquid, liquid/liquid/solid, liquid/solid/solid, etc.

As described herein, sucralose-6-esters can be prepared by (a) creating a heterogeneous mixture comprising a first phase comprising a sucrose-6-ester and a second phase comprising a chlorinating reagent; and (b) reacting the sucrose-6-ester with the chlorinating reagent.

Preferably, the sucralose-6-ester is a compound represented by the following formula (II):

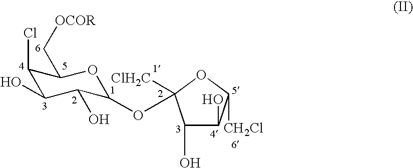

wherein R represents a substituted or unsubstituted aryl group or a substituted or unsubstituted alkyl group.

Preferably, R represents a substituted or unsubstituted phenyl group or a substituted or unsubstituted alkyl group having 1 to 4 carbon atoms. More preferably, R represents phenyl, p-methyl phenyl, methyl, ethyl, propyl, butyl or benzyl. Most preferably, R is methyl or phenyl.

Preferably, the heterogeneous mixture further comprises a non-hydrophilic solvent, such as an ester, a halogenated alkane, an aromatic, etc. These solvents may be used individually or in combination thereof. More preferably, the non-hydrophilic solvent has an appropriate boiling point, for example, ranging from about 110° C. to about 140° C., so as to facilitate reaction operation and workup process, as well as recycling of the solvent.

Examples of suitable non-hydrophilic ester solvents may include, but are not limited to, a solvent represented by the formula R⁷COOR⁸, wherein R⁷ and R⁸ are the same or different and each independently represents an alkyl group having 1 to 8 carbon atoms. More preferably, the non-hydrophilic ester solvent contains butyl acetate or n-propyl acetate. Examples of suitable non-hydrophilic halogenated alkane solvents may include, but are not limited to, 1,1,2-trichloroethane, 1,1,1,2-tetrachloroethane and 1,1,2,2-tetrachloroethylene. Examples of suitable non-hydrophilic aromatic solvents may include, but are not limited to, toluene, xylene and chlorobenzene. These solvents may be used individually or in combination thereof.

The chlorinating reagent may be any suitable reagent capable of chlorinating a sucrose-6-ester in the 4-,1′- and 6′-positions. Preferably, the chlorinating reagent contains a reagent of the Vilsmeier type, i.e., an N,N-dialkyl(chloromethaniminium) chloride represented by the following formula:

[HXC═N⁺R¹R²]Cl⁻

wherein X represents —Cl or —OPOCl₂, and R¹ and R² are the same or different, and each independently represents an alkyl group typically having 1 to 4 carbon atoms; alternatively, R¹ represents an alkyl group and R² represents a phenyl group.

A Vilsmeier reagent may be prepared by one of various methods, for example, by a reaction of a N-formyl amide, such as DMF, and an acyl chloride, such as phosgene (COCl₂), phosgene dimer (Cl—CO—O—CCl₃), phosgene trimer (CCl₃—O—CO—O—CCl₃), oxalyl chloride (Cl—CO—CO—Cl), phosphorus pentachloride (PCl₅), thionyl chloride (SOCl₂) and phosphorus oxychloride (POCl₃).

Some exemplary reaction schemes for preparation of Vilsmeier reagents are illustrated below:

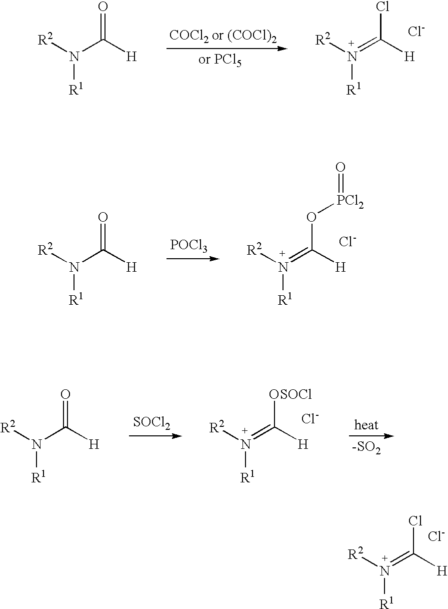

The Vilsmeier reagents can be prepared in a non-hydrophilic solvent such as those described above. An exemplary procedure is as follows. To a stirring solution of a N-formyl amide, preferably DMF, in a non-hydrophilic solvent, is added an acyl chloride, preferably, phosgene, phosgene dimer, phosgene trimer, oxalyl chloride or thionyl chloride. The acyl chloride can be added in pure form or as a solution in the non-hydrophilic solvent. This addition step can preferably be carried out at a temperature of about −5° C. or below and under an inert atmosphere such as nitrogen or argon. After the addition, the reaction mixture can be stirred at a temperature of about 10° C. or below for about 1 hour to prepare a Vilsmeier reagent. The resulting Vilsmeier reagent generally does not dissolve in such non-hydrophilic solvent at ambient temperature and hence can form a suspension. As used herein, the terms “suspension” and “dispersion” refer to a mixture including at least two phases, one of which is a liquid, the other a finely divided solid particles and/or liquid droplets dispersed in the liquid, and are used interchangeably.

The thus-obtained Vilsmeier reagent can then be used to chlorinate a sucrose-6-ester. The molar ratio of N-formyl amide/sucrose-6-ester can preferably be in the range of about 7:1 to about 20:1 and more preferably, about 9:1 to about 17:1. The molar ratio of acyl chloride/sucrose-6-ester is preferably in the range of about 6:1 to about 14:1 and more preferably, about 8:1 to about 11:1.

The obtained Vilsmeier reagent may also be separated from the reaction mixture and stored under an inert atmosphere for future use.

An exemplary chlorination process of a Vilsmeier reagent is illustrated as follows:

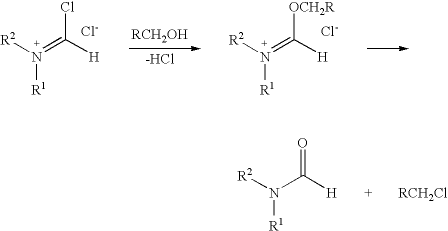

When a sucrose-6-ester is chlorinated with a Vilsmeier reagent, the reactivity of the hydroxyl groups in the sucrose-6-ester is generally in the order of 6′>4>1′>others.

To create a heterogeneous mixture of a sucrose-6-ester and a Vilsmeier reagent, the sucrose-6-ester may be added to a stirring suspension of the Vilsmeier reagent in a non-hydrophilic solvent, preferably, at about 10° C. or below and under an inert atmosphere such as nitrogen and argon. The sucrose-6-ester may be added either in pure form or as a solution in an appropriate solvent. Examples of suitable solvents may include, but are not limited to, DMF and dimethyl sulfoxide (DMSO). Preferably, the sucrose-6-ester is dissolved in DMF.

Typically, sucrose-6-esters are in the form of either syrup or solid. When a sucrose-6-ester is in the form of syrup, addition thereof may be difficult. Thus, such sucrose-6-ester can preferably be diluted in a small amount of solvent prior to addition to the Vilsmeier reagent. When a sucrose-6-ester is in the form of solid, it can be added directly or in the alternative, dissolved in a small amount of solvent and then added to the Vilsmeier reagent. Addition of a solution can simplify the operation procedure for large-scale production and thus is preferred. Typically, a sucrose-6-ester can be prepared as a solution in DMF at a concentration of about 40% to about 60% by weight based on the weight of the solvent.

Upon addition of the sucrose-6-ester to the Vilsmeier reagent, a heterogeneous system can be formed, in which a first phase can contain substantially sucrose-6-ester, a second solid phase can contain substantially the Vilsmeier reagent, and a third phase can contain substantially the non-hydrophilic solvent. Upon completion of the addition, the weigh ratio of sucrose-6-ester:solvents including the non-hydrophilic solvent and DMF in the Vilsmeier reagent can preferably be 1:6-16 and more preferably 1:8-12.

Preferably, the chlorination reaction of a sucrose-6-ester with a Vilsmeier reagent can be carried out in the presence of a phase transfer reagent (also known as “phase transfer catalyst”). The term “phase transfer reagent” is understood by those skilled in the art and generally refers to a reagent which extracts one of the reactants from one phase, cross the interface into another phase so that reaction can proceed. In the present context, the phase transfer reagent can preferably contain a quaternary ammonium salt such as a compound represented by formula R¹R²R³R⁴N⁺Cl⁻, wherein R¹, R², R³ and R⁴ are the same or different and each independently represents a substituted or unsubstituted alkyl group. Preferably, R¹, R², R³ and R⁴ each independently represents a substituted or unsubstituted alkyl group having 1 to 16 carbon atoms. Examples of suitable phase transfer reagents may include, but are not limited to, benzyltriethylammonium chloride, benzyltrimethylammonium chloride, tetrabutylammonium chloride and hexadecyl trimethylammonium chloride. The phase transfer reagent can preferably be used in an amount of about 5% to about 30% by mole based on the amount of sucrose-6-ester.

Chlorination of the sucrose-6-ester can be further effected as follows. The mixture of the sucrose-6-ester and the Vilsmeier reagent can be gradually heated over a period of time, e.g., about 1 hour, to a first elevated temperature of about 75° C. to about 95° C. The Vilsmeier reagent may begin to dissolve in the non-hydrophilic solvent at about 60° C. At about 75° C. to about 95° C., the Vilsmeier reagent may become dissolved in the non-hydrophilic solvent and the chlorination reaction of the sucrose-6-ester may proceed in a heterogeneous medium. The reaction mixture may gradually turn from milk-white color to golden yellow. The mixture can be allowed to react at this temperature, and preferably, about 80° C. to about 90° C. The reaction can be monitored, for example, by means of thin layer chromatography (TLC). As the reaction proceeds, the hydroxyl groups of the sucrose-6-ester can be partially chlorinated to produce a mixture of dichloroesters including 4,6′-dichlorosucrose-6-ester and 1′,6′-dichlorosucrose-6-ester, which can substantially dissolve in the non-hydrophilic solvent. Upon substantially complete conversion of the sucrose-6-ester to partially chlorinated products thereof, typically, in about 1.5 hours to about 3.0 hours, the reaction mixture may turn into a homogeneous system.

The reaction mixture can be further heated to a second elevated temperature of about 105° C. to about 125° C., preferably about 110° C. to about 120° C., and allowed to react at this temperature. As the reaction proceeds, the partially chlorinated sucrose-6-ester mixture can be further chlorinated to produce the corresponding sucralose-6-ester. Typically, this step may be substantially complete in about 3.0 hours to about 5.0 hours.

Upon completion of the chlorination reaction, the reaction mixture can be worked up to recover the sucralose-6-ester. Typically, the reaction mixture can be cooled to a temperature of about 10° C. or below, and then neutralized to pH about 7. For instance, the reaction mixture can be treated with an aqueous basic solution, such as a 4N aqueous NaOH solution, to pH about 10, and then treated with an acid, such as acetic acid, to pH about 7. Preferably, during the neutralization step, the temperature of the reaction mixture can be maintained at about 40° C. or below.

Optionally, the neutralized solution can be further treated with a decolorizing agent such as activated carbon, macroporous adsorbent resins, etc. Activated carbon is a preferred adsorbent because of its relatively low cost and relatively high adsorption efficiency. The mixture can then be filtered to substantially remove solid contents. The organic phase can be separated and the aqueous phase can be extracted with an organic solvent. Suitable organic solvents may include esters, chlorinated alkanes, etc. and can preferably include ethyl acetate. The organic extracts can be combined with the initial organic phase. The combined organic phases can then be concentrated under a reduced pressure to provide the sucralose-6-ester. When the extracting solvent is different from the reaction solvent (i.e., the non-hydrophilic solvent), the organic extracts and the initial organic phase can preferably be concentrated separately to facilitate recycling of the solvents respectively.

The obtained sucralose-6-ester can be deesterified by any known method, for example, by treatment with sodium methoxide/methanol or sodium ethoxide/ethanol, to prepare sucralose. Sucralose may be further purified by crystallization.

Prior to the deesterification step, the sucralose-6-ester may be optionally further purified by crystallization. For example, sucralose-6-benzoate can be crystallized from a solvent system such as petroleum ether/water, or tert-butyl methyl ether/water.

To further purify sucralose-6-acetate, crude sucralose-6-acetate can be first dissolved in water, preferably at an elevated temperature, e.g., at about 50° C. The sucralose-6-acetate content in the aqueous solution preferably ranges from about 20% wt/v to about 40% wt/v. The aqueous solution can be cooled gradually to obtain a first crystallized product. The first crystallized product can then be dissolved in ethanol, e.g., at about 50° C. The sucralose-6-acetate content in the ethanol solution is preferably about 20% wt/v to about 40% wt/v. The ethanol solution can be cooled gradually to obtain a second crystallized product, which is substantially free of the dichloro-substituted byproducts. The second crystallized product can further be dissolved in ethyl acetate, e.g., at about 50° C. The sucralose-6-acetate content in the ethyl acetate solution is preferably about 20% wt/v to about 40% wt/v. The ethyl acetate solution can then be cooled gradually to obtain sucralose-6-acetate with a relatively high purity. In this configuration, the recrystallization order can be altered. For instance, recrystallization with ethyl acetate can be carried out prior to recrystallization with ethanol. If necessary or desired, recrystallization with ethyl acetate and/or ethanol may be repeated one or more times.

The thus-obtained sucralose-6-acetate can be converted to sucralose, upon deesterification, e.g., by treatment with sodium methoxide/methanol. The reaction mixture can be neutralized with acid, such as acetic acid and strong acid ion exchange resin, filtered and concentrated to remove methanol. This sucralose, after crystallization, e.g., with ethyl acetate, can have a relatively high purity meeting the standards under the United States Pharmacopeia (USP-29th Ed.). Each of the above-mentioned crystallization steps may be repeated one or more times, if necessary or desired.

In an exemplary process, a crude sucralose-6-acetate was crystallized from water to obtain a first crystallized product. HPLC analysis of the first crystallized product (C18 column, 40:60 v/v methanol:water as eluent) showed contents of sucralose-6-acetate in 82.8% by area and six impurities having a content of greater than 0.5% by area, one of which is less polar than sucralose-6-acetate (4.6% by area), and five of which are more polar than sucralose-6-acetate (1.1%, 2.3%, 2.1%, 0.93%, and 0.53% by area, respectively). Reverse phase TLC analysis showed six impurity spots, four of which are present in an amount of greater than 0.5%, two being more polar than sucralose-6-acetate and the other two being less polar than sucralose-6-acetate.

Recrystallization of the first crystallized product with 95% v/v ethanol and ethyl acetate, respectively, afforded sucralose-6-ester having purity of 99.4% by area. Reverse phase TLC analysis did not show any impurity contents of greater than 0.5%.

On the other hand, when the first crystallized product was recrystallized with 95% v/v ethanol or ethyl acetate, but not both, the resulting sucralose-6-ester still contained impurities each having a content of greater than 0.5% by reverse phase TLC analysis.

When the sucralose-6-ester is sucralose-6-acetate, it can also be converted into 4,1′,6′-trichloro-4,1′,6′-trideoxy galactosucrose-penta-acetate (TOSPA) represented by the following formula (III), which in turn can be deacetylated to prepare sucralose:

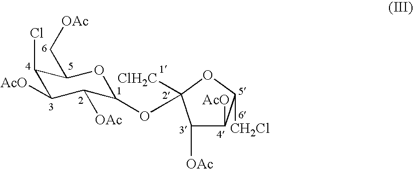

For example, sucralose-6-acetate can be treated with an acylating agent in the presence of a catalytic amount of iodine (I₂) to prepare TOSPA. Preferably, the acylating agent can contain acetic anhydride, acyl chloride, or the like. In the present context, an acylating agent can preferably be used in an amount of about 5 to about 15 equivalents based on the amount of sucralose-6-acetate. Further, I₂ can preferably be used in an amount of about 0.05 to about 0.3 equivalents based on the amount of sucralose-6-acetate. More preferably, the molar ratio of acylating agent:sucralose-6-acetate:I₂ can be about 6:1:0.1. That is, the amount of acylating agent can be about 1.5 times by mole the amount of the hydroxyl groups in sucralose-6-acetate. This reaction can be conducted in a manner similar to that described by P. Phukan in Iodine as an extremely powerful catalyst for the acetylation of alcohols under solvent-free conditions, Tetrahedron Letter, 45, 4785-87 (2004). Typically, the peracylation reaction can be substantially complete in a relatively short period of time, for example, about 0.5 hour to about 2.0 hour, at ambient temperature.

The reaction mixture can be diluted with an organic solvent. Examples of suitable organic solvents may include, but not are limited to toluene, methylene chloride, 1,2-dichloroethane, ethyl acetate, propyl acetate and butyl acetate. These solvents may be used individually or in combination thereof. Preferably, the organic solvent contains toluene. The diluted mixture can then be washed with an aqueous solution of sodium sulfite, sodium hydrogensulfite or sodium thiosulfate and water, respectively, and concentrated under a reduced pressure to an appropriate volume. The residue can then be cooled, e.g., in an ice bath, to crystallize TOSPA. Preferably, a pure TOSPA crystal seed can be added to facilitate crystallization. This crystallization step can substantially remove impurities, in particular, any tetrachloro tetradeoxy galactosucrose-tetra-acetate byproducts. If desired or necessary, this crystallization step may be repeated one or more times.

Preferably, the crystallized TOSPA can be recrystallized in a suitable solvent which preferably contains a hydrophilic organic solvent such as a solvent containing ethanol or isopropanol. This recrystallization step can substantially remove impurities, in particular, any dichloro dideoxy galactosucrose-hexa-acetate byproducts. The obtained TOSPA can have a relatively high purity. If desired or necessary, this crystallization step can be repeated one or more times.

In an alternative, TOSPA can be purified as follows. The diluted reaction mixture containing TOSPA as described above can be washed with an aqueous solution of sodium sulfite, sodium hydrogensulfite or sodium thiosulfate and water, respectively, and then concentrated to remove the solvents. A hydrophilic organic solvent such as a solvent containing ethanol or isopropanol can be added to the residue to crystallize TOSPA, which can, in turn, be recrystallized from a non-hydrophilic organic solvent such as a solvent containing toluene. If desired or necessary, each of the crystallization steps with hydrophilic and non-hydrophilic organic solvents may be repeated one or more times.

As described herein, the combination use of hydrophilic and non-hydrophilic organic solvents can allow efficient removal of both dichloro dideoxy galactosucrose-hexa-acetate byproducts and tetrachloro tetradeoxy galactosucrose-tetra-acetate byproducts. In comparison, crude TOSPA, which has been subjected to crystallization for four times using ethanol, may still contain byproducts including tetrachloro tetradeoxy galactosucrose-tetra-acetate. Further, crude TOSPA, which has been subjected to crystallization for four times using toluene, may still contain byproducts including dichloro dideoxy galactosucrose-hexa-acetates.

The substantially pure TOSPA can lead to sucralose (TGS) in relatively high purity, which can significantly simplify sequential purification procedures.

TOSPA can be deacetylated, for example, by the treatment of an alkali metal/alcohol mixture, such as sodium methoxide/methanol or sodium ethoxide/ethanol, to prepare sucralose. Sucralose can be recovered by the following workup procedures. The reaction mixture can be neutralized with an acid, such as acetic acid, to pH about 7, and then concentrated under a reduced pressure to remove the alcohol solvent. The residue can be dissolved in water and extracted with a water-immiscible organic solvent, such as a solvent containing ethyl acetate, for several times. The combined organic extracts can be washed with water, dried over a drying reagent, such as anhydrous magnesium sulfate, filtered to remove solid contents, and concentrated. The resulting solution can then be cooled, e.g., in an ice bath, to crystallize sucralose. The obtained crystalline sucralose can have a purity of greater than 99%. If desired, the resulting sucralose may be crystallized, e.g. using purified water.

The present invention is further illustrated by the following specific examples but is not limited hereto.

### EXAMPLES

Unless specified, all the commercially available materials were used herein without further purification. All the temperature measurements were uncorrected.

### Comparative Example 1

### Preparation of sucralose 6-benzoate

Sucralose 6-benzoate was prepared according to U.S. Pat. Nos. 5,023,329 and 4,980,463 as follows.

A reaction vessel was charged with sucrose (55.5 g, 0.162 mol), dibutyltin oxide (42.5 g) and DMF (195 mL). The mixture was heated to 85° C. to 90° C. until all the solids were substantially dissolved, and cyclohexane (65 mL) was added thereto. The resulting mixture was heated to reflux at 90° C. to 95° C. and maintained at the temperature for 6 hours while water being removed from the reaction mixture. The reaction mixture was cooled to 0° C. using an ice bath. Benzoic anhydride (42.5 g, 90% purity) was added and the reaction mixture was allowed to warm naturally to ambient temperature and stirred overnight. TLC analysis (CHCl₃:MeOH:H₂O 15:10:1 by volume) indicated that the reaction was complete.

The reaction mixture was filtered and water (16 mL) was added to the filtrate. The filtrate was then extracted with cyclohexane (150 mL×3) to remove tin compounds. The DMF-containing phase was concentrated to obtain a residue, which was then dissolved in methanol (150 mL). The resulting solution was decolorized with activated carbon (8 g), filtered and concentrated. The resulting residue was dissolved in methylene chloride (200 mL) upon heating, and then allowed to cool naturally while stirring. Crystallization afforded crude sucralose 6-benzoate product. After drying, the crude sucrose 6-benzoate product was dissolved in methylene chloride (150 mL) upon heating, and then allowed to cool naturally to afford a crystalline product. The crystalline product was separated and dried to provide sucrose 6-benzoate (68 g).

To stirring DMF (550 mL) at −10° C. to −15° C. and under nitrogen atmosphere was slowly added oxalyl chloride (192 mL) for a period of 2 hours. The reaction temperature was controlled at −5° C. or below. The reaction mixture was allowed to stir for 20 minutes, and sucrose-6-benzoate (52 g in 72 g of DMF) was added dropwise. The mixture was sequentially heated to 60° C. and maintained at this temperature for 20 minutes, heated to 80° C. and maintained at this temperature for 60 minutes, and heated to 110° C. and maintained at 110° C. to 115° C. for 3.5 hours.

The reaction mixture was then cooled to 10° C. or below, and treated with a 4N aqueous NaOH solution (480 g) to obtain pH 10.0. The mixture was stirred for 10 minutes and then acetic acid was added thereto to obtain pH 7.1. Activated carbon (6 g) was added to the mixture, which was then filtered and concentrated to afford a syrup. This syrup was dissolved in water (600 mL) and ethyl acetate (400 mL) upon stirring. The aqueous phase was separated and extracted with ethyl acetate (200 mL×3). The combined organic phases were washed with a saturated aqueous NaCl solution (200 mL) and water (200 mL), respectively, decolorized with activated carbon (9 g) and dried over anhydrous magnesium sulfate. The dried solution was filtered and concentrated to afford TGS-6-benzoate syrup (84 g).

The thus-obtained syrup (84 g) was dispersed in water (200 mL) at 60° C. upon stirring. The dispersion was cooled to 50° C. and methyl tert-butyl ether (200 mL) was added thereto. The mixture was stirred to cool to crystallize. After stirring at 25° C. for 1 hour, the crystalline product was separated, washed with ethyl tert-butyl ether twice (100 mL and 60 mL, respectively), and dried under vacuum to afford TGS-6-benzoate (40 g). TLC analysis indicated the presence of at least one byproduct, which spot was below the TGS-6-benzoate spot.

A portion of the resulting TGS-6-benzoate (5 g) was dissolved in methanol (15 mL) upon heating and water (15 mL) was added thereto. The mixture was stirred to cool to ambient temperature and then cooled in a refrigerator for 60 minutes. The crystallized product was separated and washed with ice-cooled methanol/water (1:1 by volume, 10 mL), and dried under vacuum to afford TGS-6-benzoate. TLC analysis did not show other byproducts.

### Example 1

### Preparation of sucralose 6-benzoate

A reaction vessel was charged with benzyl triethylammonium chloride (2.0 g, 8.7 mmol), 1,1,2-trichloroethane (120 mL) and DMF (30 mL) under nitrogen atmosphere. The resulting mixture was cooled to −5° C. Oxalyl chloride (28.3 mL in 28 mL of 1,1,2-trichloroethane) was gradually added to the mixture for about one hour and the reaction temperature was controlled at 0° C. The reaction mixture turned a white suspension. Sucrose-6-benzoate (13.4 g, 30 mmol in 15 mL of DMF) was added to the reaction mixture. After the addition, the reaction mixture was allowed to warm naturally to 38° C., and was then sequentially heated to 60° C. and maintained at the temperature for 30 minutes, heated to 85° C. and maintained at the temperature for 1 hour, and heated to 102° C. and maintained at the temperature for 2 hours.

The reaction mixture was cooled to 20° C. and treated with a 4N aqueous NaOH solution to obtain pH 9.5, followed by acetic acid to pH 7.0. The resulting mixture was decolorized with activated carbon (3 g) and filtered. The aqueous phase was separated and extracted with methylene chloride (50 mL×5). The combined organic phases were washed with a 5% aqueous NaCl solution (150 mL) and water (150 mL), respectively, and concentrated to afford a syrup (14.5 g).

The obtained syrup was subjected to fast column chromatography (silica gel, 100 g), eluted with CHCl₃ (100 mL), CHCl₃:MeOH (10:1 by volume, 100 mL) and CHCl₃:MeOH (5:1 by volume, 150 mL), respectively, to afford another syrup (5.2 g). To this syrup was dispersed in water (10 mL), upon stirring and heating. Petroleum ether (10 mL) was then added to produce a crystalline product. The crystalline product was separated, washed with petroleum ether (10 mL×2), and concentrated under vacuum to afford sucralose 6-benzoate (2.5 g), which was found by TLC analysis to be the same as the sample obtained in Comparative Example 1.

### Example 2

### Preparation of sucralose 6-benzoate

The process described in Example 1 was repeated, except benzyl trimethylammonium chloride (1.6 g) was used instead of benzyl triethylammonium chloride. 12.5 g of sucralose 6-benzoate was obtained, prior to fast column chromatography.

### Example 3

### Preparation of sucralose 6-benzoate

The process described in Example 1 was repeated, except triphosgene (41.6 g, 105 mmol) was used instead of oxalyl chloride, and 0.67 g of benzyl triethylammonium chloride was used. The reaction afforded 14.5 g of sucralose 6-benzoate syrup, which, upon fast column chromatography, afforded 3.5 g of sucralose 6-benzoate.

### Example 4

### Preparation of sucralose 6-acetate

Under nitrogen atmosphere, a reaction vessel was charged with benzyl triethylammonium chloride (2 g, 8.7 mmol), n-propyl acetate (120 mL) and DMF (30 mL). The resulting mixture was cooled to 15° C. or below and SOCl₂ (28.3 mL) was added thereto for a period of 10 minutes. The mixture turned a white suspension. To the mixture was added sucrose-6-acetate (11.5 g, 30 mmol in 11 mL of DMF). The reaction mixture was heated to 54° C. and then 65° C., and maintained at this temperature for 30 minutes. At this stage, the reaction mixture was pale yellow dispersion. The reaction mixture continued to be heated to 80° C. to 85° C., and maintained at this temperature for 1 hour. After 15 minutes at this temperature, the reaction mixture turned a clearer red-brown dispersion. The reaction mixture was then heated to 95° C., and maintained at 92° C. to 97° C. for 3.5 hours. After 25 minutes at this temperature, the reaction mixture became a clear red-brown solution. Further, the reaction mixture was heated to 105° C. and allowed to react at this temperature for 3 hours.

The reaction mixture was cooled to 18° C. and water (20 mL) was added thereto. A 4N aqueous NaOH solution was added to the mixture to obtain pH 9.5. The reaction mixture was stirred for 10 minutes and acetic acid was added thereto to obtain pH 7.0. To the mixture was added water (180 mL). The mixture was decolorized with activated carbon (3 g) and filtered. The aqueous phase was separated and extracted with ethyl acetate (100 mL×5). The combined organic phases were dried over anhydrous magnesium sulfate, filtered and concentrated to afford a syrup (13.5 g). TLC analysis (CHCl₃:MeOH 5:1 by volume) indicated that the product primarily comprised of TGS-6-Ac.

### Example 5

### Preparation of sucralose 6-acetate

Under nitrogen atmosphere, a reaction vessel was charged with benzyl triethylammonium chloride (6 g, 26.3 mmol), n-butyl acetate (800 mL) and DMF (228 mL). The resulting mixture was cooled to 0° C. or below and SOCl₂ (287 g) was added thereto for a period of 20 minutes. During the addition, the temperature of the reaction mixture was controlled under 20° C. The mixture was stirred at ambient temperature for 30 minutes and turned into a white suspension. To the mixture was added sucrose-6-acetate (103 g, 0.268 mol in 103 mL of DMF). The reaction mixture was heated to 70° C. to 75° C. and maintained at this temperature for 30 minutes. Then, the reaction mixture continued to be heated to 85° C. to 90° C., and maintained at this temperature for 1 hour. Thereafter, the reaction mixture was heated to 95° C. to 98° C., and maintained at this temperature for 1.5 hours. Further, the reaction mixture was heated to 115° C. and allowed to react at this temperature for 3.5 hours.

The reaction mixture was cooled to 5° C. and a 4N aqueous NaOH solution (530 mL) was added thereto to obtain pH 9.5. During the addition of the NaOH solution, the temperature of the reaction mixture was controlled under 35° C. The reaction mixture was stirred for 10 minutes and acetic acid was added thereto to obtain pH 7.0. The aqueous phase of the mixture was separated and decolorized with activated carbon (5 g) and filtered. The aqueous phase was then extracted with ethyl acetate (500 mL×1 and 300 mL×5). The combined organic phases were decolorized with activated carbon (10 g), filtered, and concentrated to afford a TGS-6-Ac syrup (101 g).

To the resulting syrup was added water (200 mL) and the mixture was stirred to dissolve at 50° C. The mixture was then cooled to 30° C. and diethyl ether (10 mL) was added thereto. The resulting mixture was cooled to 20° C. and stirred to crystallize for 6 hours. The resulting crystals were filtered and washed with water (75 mL×2), and dried under vacuum to afford TGS-6-Ac crystals (47 g).

### Example 6

### Purification of TGS-6-Ac

The crude TGS-6-Ac (100 g) was heated to dissolve in 95% v/v ethanol (200 mL) and decolorized with activated carbon. The mixture was filtered and naturally cooled to ambient temperature to crystallize while stirring. After 3 hours, the crystals were collected and the filter flask was washed with a small amount of 95% v/v ethanol to afford wet crystals (90.3 g).

The resulting wet crystals were heated to dissolve in anhydrous ethyl acetate (220 mL). The mixture was cooled naturally to ambient temperature and stirred for 3 hours. The resulting crystals were collected and washed with a small amount of ethyl acetate, and dried under vacuum (45° C. to 50° C., 0.09 MPa), to afford TGS-6-Ac having a purity of 99.4%. Reverse phase TLC analysis did not detect any impurities having contents of greater than 0.5%.

### Example 7

### Preparation of sucralose from TGS-6-Ac

Under nitrogen atmosphere, the purified TGS-6-Ac obtained in Example 6 (20 g) was dissolved in anhydrous methanol (200 mL). The resulting mixture was stirred at ambient temperature and sodium methoxide (0.25 g) was added thereto. The reaction mixture was stirred for 2 hours. TLC analysis (CHCl₃:MeOH 3:1 v/v) indicated that the reaction was complete. The resulting mixture was neutralized with strong acid ion exchange, decolorized with active carbon and filtered. The filtrate was subjected to a reduced pressure to remove about 70% of methanol under 45° C. To the resulting mixture was added ethyl acetate (200 mL). The mixture was again subjected to a reduced pressure to remove the remaining methanol. At this stage, the mixture contained ethyl acetate as the solvent. After removal of 80 mL to 100 mL of ethyl acetate under 50° C., the mixture was cooled slowly to ambient temperature to afford sucralose (13 g). External standard analysis of the resulting product indicated a content of 100% with impurity levels with the USP-29 requirements.

### Comparative Example 2

### Preparation of 4,1′,6′-trichloro-4,1′,6′-trideoxy galactosucrose-penta-acetate (TOSPA)

TOSPA was prepared according to U.S. Pat. Nos. 4,362,869 and 4,783,526 as follows.

A reaction vessel under nitrogen atmosphere was charged with DMF (140 mL), sucrose (34.2 g, 100 mmol) and triethylamine (69 mL, 495 mmol). The resulting mixture was heated to 45° C. and stirred for 25 minutes. 4-(Dimethylamino) pyridine (DMAP) (1.47 g) was added to the reaction mixture. The resulting mixture was stirred for 5 minutes and triphenylchloromethane (92 g, 330 mmol) was added batchwise for a period of 2.5 hours to 3.0 hours, during which the reaction temperature was controlled at 35° C. to 40° C. After the addition, the reaction mixture was allowed to stir at ambient temperature overnight. TLC analysis (CHCl₃:MeOH 10:1 by volume) showed that all the starting sucrose (Rf 0.05) was consumed. The 6,1′,6′-tri-O-tritylsucrose product spot (Rf 0.35) showed color when exposed to 5% H₂SO₄/EtOH.

To the above mixture under nitrogen atmosphere was added acetic anhydride (57 mL, 600 mmol). The resulting mixture was heated to 65° C. to 70° C. and stirred for 4.5 hours to 5.0 hours. TLC analysis (CHCl₃:MeOH 10:1 by volume) showed that all 6,1′,6′-tri-O-tritylsucrose was consumed. The 6,1′,6′-tri-O-tritylsucrose penta-acetate (TRISPA) product had Rf of 0.4 (hexane:EtOAc 3:1 by volume).

The reaction mixture was cooled to ambient temperature and poured into a mixture of water (350 g) and ice (100 g). The resulting mixture was stirred for 5 minutes to crystallize and filtered. The filter flask was washed with water (100 mL×4) and methanol (50 mL×2), respectively. A wet solid product (200 g) was obtained.

The resulting wet solid was dissolved in acetone (300 mL) upon heating. The solution was then cooled to crystallize. The crystalline product was separated and dried under vacuum for 4 hours to provide white TRISPA crystals (37 g). The melting point of TRISPA was measured using Shanghai Shenguang WRR melting point apparatus with the thermometer uncorrected to be 229.8° C. to 232.0° C.

The thus-obtained TRISPA (50 g, 39 mmol) was dissolved in CH₂Cl₂ (150 mL) upon stirring for about 30 minutes at ambient temperature and under nitrogen atmosphere. To this solution was added a solution of anhydrous HCl in methanol (0.75 M, 15 mL). The reaction mixture was allowed to react for 4.5 hours. TLC analysis (CH₂Cl₂:MeOH 20:1 by volume) showed that all TRISPA was consumed.

Nitrogen was bubbled into the reaction mixture to remove HCl gas. The mixture was then subjected to vacuum distillation to remove CH₂Cl₂, thereby providing purple solid. The purple solid was dissolved in methanol (5% water, 150 mL) upon stirring for 30 minutes. The resulting solution was cooled in a refrigerator to crystallize, filtered to remove solid triphenylmethanol derivatives. The filter flask was washed with ice-cooled methanol (5% water, 20 mL×2). The filtrate was dried under vacuum, and again after addition of toluene (20 mL). The residue was dissolved in ethyl ether (200 mL) and cooled in a refrigerator overnight to crystallize. The crystalline product was separated and dried under vacuum to provide white 2,3,4,3′,4′-penta-O-acetylsucrose (4-PAS) (20.5 g), m.p. 94.2° C. to 96.7° C.

The thus-obtained 4-PAS (21.4 g, 39 mmol) was dissolved in ethyl acetate (33 mL) under nitrogen atmosphere, and tert-butylamine (2.5 mL) was added thereto. The resulting mixture was reacted at 45° C. to 50° C. for 6 hours and concentrated. The residue was dissolved in toluene (10 mL) and cooled to 0° C., and n-hexane (60 mL) was added dropwise thereto. The mixture was stirred for 3 hours, filtered and dried to afford 6-PAS (16.7 g, yield 78%), m.p. 151.7° C. to 152.9° C.

The thus-obtained 6-PAS was dissolved in toluene (160 mL) and DMF (21.2 g, 0.29 mol) under nitrogen atmosphere and then cooled to −15° C. To the mixture was slowly added oxalyl chloride (14.3 mL, 163 mmol in 60 mL of methanol) for a period of 60 minutes. The mixture was warmed naturally to ambient temperature and stirred for 15 minutes, and then heated to reflux under nitrogen atmosphere for 5 hours to 6 hours. TLC analysis (CHCl₃:MeOH 20:1 by volume) showed that all 6-PAS was consumed.

The reaction mixture was then decolorized with activated carbon for 20 minutes and filtered. The solid was washed with toluene. The combined organic phases were washed with 5% aqueous NaHCO₃ (100 mL) and water (100 mL), respectively, concentrated and crystallized with toluene (20 mL) to afford TOSPA (17.2 g), m.p. 89.5° C. to 90.7° C.

### Example 8

### Preparation of 4,1′,6′-trichloro-4,1′,6′-trideoxy galactosucrose-penta-acetate (TOSPA)

Under nitrogen atmosphere, a reaction vessel was charged with benzyl triethylammonium chloride (2 g, 8.7 mmol), 1,1,2-trichloroethane (120 mL) and DMF (30 mL). The resulting mixture was cooled to −5° C. and oxalyl chloride (28.3 mL in 28 mL of 1,1,2-trichloroethane) was added thereto for a period of 30 minutes, during which the reaction temperature was controlled at 0° C. To the mixture was added sucrose-6-acetate (11.5 g, 30 mmol in 11 mL of DMF). The reaction mixture was warmed naturally to 36° C. and then sequentially heated to 60° C. and maintained at this temperature for 60 minutes, heated to 85° C. and maintained at 80° C. to 90° C. for 1 hour, and heated to 105° C. and maintained at 105° C. to 110° C. for 3 hours.

The reaction mixture was then cooled to 20° C. and treated with a 4N aqueous NaOH solution to obtain pH 9.5. The reaction mixture was stirred for 10 minutes and acetic acid was added thereto to obtain pH 7.0. The mixture was subjected to vacuum to remove 1,1,2-trichloroethane. The residue was dissolved in water (100 mL), which solution was then decolorized with activated carbon (3 g) and filtered. The filtrate was extracted with ethyl acetate (250 mL×1 and 100 mL×3). The combined organic phases were dried over anhydrous magnesium sulfate, filtered and concentrated to afford a syrup (18.5 g). TLC analysis (CHCl₃:MeOH 5:1 by volume) indicated that the product primarily comprised of TGS-6-Ac.

To the resulting TGS-6-Ac (18.5 g) under nitrogen atmosphere was added pyridine (1.25 mL) and acetic anhydride (50 mL). The mixture was allowed to react at 45° C. to 50° C. for 5 hours. TLC analysis indicated that the reaction was complete. The reaction mixture was diluted with toluene (130 mL) and washed with water (80 mL×6). The organic phase was decolorized with activated carbon (3 g), filtered and concentrated to afford a syrup (12 g), which was found by TLC chromatography to be the same as TOSPA obtained in Comparative Example 2.

### Example 9

### Preparation of 4,1′,6′-trichloro-4,1′,6′-trideoxy galactosucrose-penta-acetate (TOSPA)

Under nitrogen atmosphere, a reaction vessel was charged with benzyl triethylammonium chloride (2 g, 8.7 mmol), 1,1,2-trichloroethane (104 mL) and DMF (20.8 mL). The resulting mixture was cooled to 10° C. or below and SOCl₂ (20.8 mL) was added thereto for a period of 10 minutes. To the mixture was added sucrose-6-acetate (11.5 g, 30 mmol in 11 mL of DMF). The resulting mixture was sequentially heated to 80° C. and maintained at this temperature for 60 minutes, heated to 90° C. and maintained at this temperature for 1 hour, and heated to 110° C. and maintained at this temperature for 3 hours.

The reaction mixture was then cooled to 8° C. and treated with a 4N aqueous NaOH solution to obtain pH 9.5. The reaction mixture was stirred for 10 minutes and acetic acid was added thereto to obtain pH 7.0. To the mixture was added water (60 mL) and CHCl₃ (60 mL). The resulting mixture was decolorized with activated carbon (3 g) and filtered. The aqueous phase was extracted with ethyl acetate (100 mL×1 and 50 mL×2). The combined organic phases were dried over anhydrous magnesium sulfate, filtered and concentrated to afford a syrup (4.5 g), which was found by TLC analysis (CHCl₃:MeOH 5:1 by volume) to primarily comprise of TGS-6-Ac.

To the resulting TGS-6-Ac (2.5 g) under nitrogen atmosphere was added pyridine (2 drops) and acetic anhydride (10 mL). The resulting mixture was allowed to stir at ambient temperature overnight. TLC analysis (ethyl ether:petroleum ether 4:1 by volume) indicated that the reaction was complete. The reaction mixture was diluted with toluene (30 mL) and water (20 mL), and treated with a 4N aqueous NaOH solution to obtain pH 6.5. The organic phase was separated and concentrated to afford TOSPA syrup (3 g).

The resulting TOSPA syrup was dissolved in ethanol (9 mL) upon heating and cooled naturally. TOSPA crystal seeds were added to the resulting solution to crystallize. The crystalline product was separated, washed with ice-cooled EtOH (10 mL), filtered and dried under vacuum to provide TOSPA crystals (1.5 g).

### Example 10

### Preparation of 4,1′,6′-trichloro-4,1′,6′-trideoxy galactosucrose-penta-acetate (TOSPA)

Under nitrogen atmosphere, a reaction vessel was charged with benzyl triethylammonium chloride (2 g, 8.7 mmol), n-butyl acetate (104 mL) and DMF (20.8 mL). The resulting mixture was cooled to 5° C. or below and SOCl₂ (20.8 mL) was added thereto for a period of 15 minutes. The resulting mixture was a white suspension. To the mixture was added sucrose-6-acetate (11.5 g, 30 mmol in 14 mL of DMF). The added sucrose-6-acetate was immiscible with the reaction medium and partially stuck to reaction vessel and stirrer. The resulting mixture was sequentially heated to 80° C. and maintained at this temperature for 60 minutes, heated to 90° C. and maintained at this temperature for 1 hour, and heated to 100° C. and maintained at this temperature for 3 hours.

The reaction mixture was then cooled to 20° C. and water (20 mL) was added. The mixture was treated with a 4N aqueous NaOH solution to obtain pH 9.5. The reaction mixture was stirred for 10 minutes and then treated with acetic acid to obtain pH 7.0. The organic phase was separated and ethyl acetate (50 mL) was added thereto. The resulting mixture was decolorized with activated carbon (2 g) and filtered. The filtering flask was washed with water (10 mL). The aqueous phase was extracted with ethyl acetate (50 mL×3). The combined organic phases were dried over anhydrous magnesium sulfate, filtered and concentrated to afford a syrup (11.5 g). TLC analysis (CHCl₃:MeOH 5:1 by volume) indicated that the product primarily comprised of TGS-6-Ac.

To the resulting TGS-6-Ac (11.5 g) under nitrogen atmosphere was added pyridine (0.5 mL) and acetic anhydride (45 mL). The mixture was allowed to stir at ambient temperature overnight. TLC analysis (ethyl ether:petroleum ether 4:1) indicated that the reaction was complete. The reaction mixture was diluted with toluene (200 mL) and water (50 mL), and treated with 4N aqueous NaOH solution to obtain pH 7.2. The organic phase was separated, washed with water (100 mL×2) and concentrated to afford a syrup.

The resulting syrup was dissolved in ethanol (60 mL) upon heating and then cooled naturally to crystallize. The crystalline product was separated, washed with ice-cooled EtOH (10 mL), filtered and dried under vacuum to provide crystalline TOSPA (2 g).

### Example 11

### Preparation of 4,1′,6′-trichloro-4,1′,6′-trideoxy galactosucrose-penta-acetate (TOSPA)

Under nitrogen atmosphere, a reaction vessel was charged with benzyl triethylammonium chloride (6 g, 26.3 mmol), n-butyl acetate (800 mL) and DMF (228 mL). The resulting mixture was cooled to −15° C. or below and oxalyl chloride (250 mL) was added thereto for a period of 2.5 hours. The resulting mixture was a white suspension. To the mixture was added sucrose-6-acetate (103 g, 268 mmol in 103 mL of DMF). The resulting mixture was sequentially heated to 80° C. and maintained at this temperature for 60 minutes, heated to 90° C. and maintained at this temperature for 1 hour, and heated to 110° C. and maintained at this temperature for 3.5 hours.

The reaction mixture was cooled to 5° C. and treated with a 4N aqueous NaOH solution (530 mL) to obtain pH 9.5. The reaction mixture was stirred for 10 minutes and then treated with acetic acid to obtain pH 7.0. The aqueous phase was separated and decolorized with activated carbon (5 g) and filtered. The aqueous phase was separated and extracted with ethyl acetate (500 mL×1 and 300 mL×5). The combined organic phases were decolorized with activated carbon (10 g) and filtered, dried over anhydrous magnesium sulfate, filtered and concentrated. The residue was dissolved with toluene (50 mL) and concentrated to afford TGS-6-Ac syrup (59 g).

To the resulting TGS-6-Ac (59 g) under nitrogen atmosphere was added iodine (3.4 g) and acetic anhydride (80 mL). The mixture was allowed to stir at ambient temperature for 60 minutes. TLC analysis (ethyl ether:petroleum ether 4:1 by volume) indicated that the reaction was complete. The reaction mixture was diluted with toluene (500 mL) and a 5% Na₂SO₃ aqueous solution (200 mL). To the organic phase was added water (300 mL). The resulting solution was treated with a 4N aqueous NaOH solution to obtain pH 6.8. The organic phase was separated, washed with water (300 mL×2), decolorized with activated carbon (10 g) and filtered, concentrated to afford TOSPA syrup (70 g).

The thus-obtained TOSPA syrup was dissolved in ethanol (300 mL) upon heating to 50° C., and then cooled naturally to ambient temperature to crystallize overnight. The crystalline product was separated by filtration. The filter flask was washed with ice-cooled EtOH (30 mL). The product was dried under vacuum to provide crystalline TOSPA (28 g).

### Example 12

### Purification of TOSPA

Crude TOSPA (39 g) was dissolved in ethanol (350 mL) upon heating at 50° C. TLC analysis of the crude TOSPA showed two byproduct spots, one above the TOSPA spot and the other below. The solution was cooled to 45° C. Needlelike crystals were formed. Upon cooling to 40° C., the crystals grew larger in diameter and became white opaque. The mixture was naturally cooled to ambient temperature overnight and filtered. The crystals were washed with ethanol (20 mL) and dried under vacuum at 50° C. to provide TOPSA (29 g). TLC analysis of the thus-obtained crystalline TOSPA showed that the byproduct spot below the TOSPA spot disappeared. However, the byproduct spot above the TOSPA spot was still present. Recrystallization with ethanol three times could not remove this byproduct(s).

### Example 13

### Purification of TOSPA

Crude TOSPA (20 g) was dissolved in toluene (170 mL) upon heating at 62° C. TLC analysis of the crude TOSPA showed two byproduct spots, one above the TOSPA spot and the other below. The solution was naturally cooled to ambient temperature and then cooled in a water bath overnight. The crystalline product was separated by filtration, washed with toluene (10 mL) and dried under vacuum at 50° C. to provide TOSPA (15 g). TLC analysis of the thus-obtained crystalline TOSPA showed that the byproduct spot above the TOSPA spot disappeared.

### Example 14

### Preparation of Sucralose from TOSPA

Purified TOSPA (20 g) was dissolved in anhydrous methanol (200 mL) upon heating at 45° C. under nitrogen atmosphere. The resulting solution was cooled in a water bath to ambient temperature and sodium methoxide (0.9 g) was added. An exothermic reaction caused the temperature to increase. The reaction mixture was cooled in a water bath to 28° C. and stirred at ambient temperature for 1.5 hours. TLC analysis (CHCl₃:MeOH 4:1 by volume) indicated that the reaction was complete.

The reaction mixture was then cooled to below 20° C. and treated with an aqueous acetic acid solution to obtain pH 7.0. The mixture was concentrated under vacuum at below 50° C. to afford TGS syrup (21.5 g). This syrup was dissolved in water (15 mL) and extracted with ethyl acetate (15 mL×3). The combined organic phases were dried over anhydrous magnesium sulfate and filtered. The water content in the organic phases was measured, which did not meet the qualifications. The filtrate was concentrated and the resulting residue dissolved in ethyl acetate (40 mL), which meets the water content qualifications, upon stirring at 60° C. using a water bath. The resulting solution was naturally cooled and stirred overnight, and filtered. The crystalline product was washed with cold anhydrous ethyl acetate (2 mL) and dried under vacuum to provide white TGS crystals (8.5 g), with HPLC purity of 99.91% by area.

From the foregoing description and illustration of this invention it is apparent that various modifications may be made to produce similar results. It is the desire of the applicants not to be bound by the description of this invention as contained in the specification, but to be bound only by the claims as appended hereto.

All of the above-mentioned references are herein incorporated by reference in their entirety to the same extent as if each individual reference was specifically and individually indicated to be incorporated herein by reference in its entirety.

What is claimed is:

1. A process for preparing a sucralose-6-ester comprising:
(a) creating a heterogeneous mixture comprising a first phase comprising a sucrose-6-ester and a second phase comprising a chlorinating reagent; and
(b) reacting the sucrose-6-ester with the chlorinating reagent, to prepare a sucralose-6-ester.

2. The process of claim 1, wherein the sucralose-6-ester is a compound represented by the following formula (II): 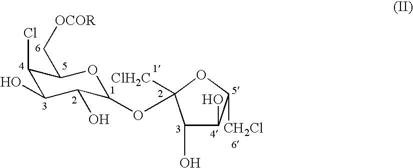
wherein R represents a substituted or unsubstituted phenyl group or a substituted or unsubstituted alkyl group having 1 to 4 carbon atoms.

3. The process of claim 2, wherein R represents methyl or phenyl.

4. The process of claim 1, wherein the chlorinating reagent is a Vilsmeier reagent represented by the following formula:
[HXC═N⁺R¹R²]Cl⁻
wherein X represents —Cl or —OPOCl₂, and R¹ and R² are the same or different, and each independently represents an alkyl group having 1 to 4 carbon atoms; or R¹ represents an alkyl group and R² represents a phenyl group.

5. The process of claim 1, wherein the heterogeneous mixture further comprises a non-hydrophilic solvent.

6. The process of claim 5, wherein the non-hydrophilic solvent comprises at least one selected from the group consisting of esters, halogenated alkanes and aromatics.

7. The process of claim 6, wherein the non-hydrophilic solvent is at least one selected from the group consisting of a solvent represented by the formula R⁷COOR⁸, wherein R⁷ and R⁸ are the same or different and each independently represents an alkyl group having 1 to 8 carbon atoms; 1,1,2-trichloroethane; 1,1,1,2-tetrachloroethane; 1,1,2,2-tetrachloroethylene; toluene; xylene and chlorobenzene.

8. The process of claim 1, wherein the heterogeneous mixture further comprises a phase transfer agent.

9. The process of claim 8, wherein the phase transfer agent comprises a compound represented by formula R¹R²R³R⁴N⁺Cl⁻, wherein R¹, R², R³ and R⁴ are the same or different, and each independently represents a substituted or unsubstituted alkyl group having 1 to 16 carbon atoms.

10. The process of claim 1, wherein the sucrose-6-ester is initially dissolved in a solvent.

11. The process of claim 10, wherein the solvent comprises N,N-dimethyl formamide or dimethyl sulfoxide.

12. The process of claim 1, wherein the creating (a) is carried out at about 10° C. or below and/or in an inert atmosphere.

13. The process of claim 1, wherein the reacting (b) comprises:
(i) heating the reaction mixture obtained in the creating (a) to a first elevated temperature of about 75° C. to about 95° C.; and
(ii) further heating the reaction mixture obtained in the heating (i) to a second elevated temperature of about 105° C. to about 125° C.

14. The process of claim 13, further comprising at least one selected from the group consisting of:
(A) maintaining the temperature of about 75° C. to about 95° C. for a period of about 1.5 hours to about 3 hours, following the heating (i) and prior to the heating (ii), and
(B) maintaining the temperature of about 105° C. to about 125° C. for a period of about 3 hours to about 5 hours, following the heating (ii).

15. The process of claim 13, wherein the first elevated temperature is in the range of about 80° C. to about 90° C.

16. The process of claim 13, wherein the second elevated temperature is in the range of about 110° C. to about 120° C.

17. A process for preparing 4,1′,6′-trichloro-4,1′,6′-trideoxy galactosucrose-penta-acetate represented by the following formula (III): 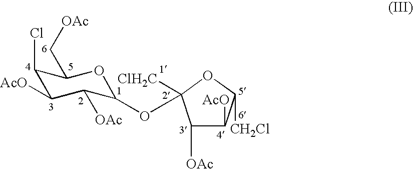
comprising:
(a) creating a heterogeneous mixture comprising a first phase comprising sucrose-6-acetate and a second phase comprising a chlorinating reagent;
(b) reacting the sucrose-6-acetate with the chlorinating reagent, to prepare sucralose-6-acetate; and
(c) reacting the sucralose-6-acetate with an acylating agent in the presence of a catalytic amount of I₂, to prepare 4,1′,6′-trichloro-4,1′,6′-trideoxy galactosucrose-penta-acetate.

18. The process of claim 17, wherein in the reacting (c), the amount of I₂ is about 0.05 to about 0.3 equivalents based on the amount of the sucralose-6-acetate.

19. The process of claim 17, further comprising purifying 4,1′,6′-trichloro-4,1′,6′-trideoxy galactosucrose-penta-acetate obtained in the reacting (c).

20. The process of claim 19, wherein the purification comprises:
(i) crystallizing crude 4,1′,6′-trichloro-4,1′,6′-trideoxy galactosucrose-penta-acetate obtained in the step (c) in a non-hydrophilic organic solvent.

21. The process of claim 20, wherein the non-hydrophilic organic solvent comprises toluene.

22. The process of claim 20, further comprising (ii) crystallizing 4,1′,6′-trichloro-4,1′,6′-trideoxy galactosucrose-penta-acetate in a hydrophilic organic solvent.

23. The process of claim 22, wherein the hydrophilic organic solvent comprises ethanol or isopropanol.

24. A process for purifying sucralose-6-ester, comprises:
(a) crystallizing crude sucralose-6-ester in ethanol to obtain a first crystallized product; and
(b) recrystallizing the first crystallized product in ethyl acetate to obtain purified sucralose-6-ester, or
(a′) crystallizing crude sucralose-6-ester in ethyl acetate to obtain a first crystallized product; and
(b′) recrystallizing the first crystallized product in ethanol to obtain purified sucralose-6-ester.

25. The process of claim 24, further comprising crystallizing the crude sucralose-6-ester in water, prior to crystallizing (a).

26. A process for preparing sucralose, comprising:
(a) creating a heterogeneous mixture comprising a first phase comprising a sucrose-6-ester and a second phase comprising a chlorinating reagent;
(b) reacting the sucrose-6-ester with the chlorinating reagent, to prepare a sucralose-6-ester; and
(c) deesterifying the sucralose-6-ester, to prepare sucralose.

27. A process for preparing sucralose, comprising:
(a) creating a heterogeneous mixture comprising a first phase comprising a sucrose-6-acetate and a second phase comprising a chlorinating reagent;
(b) reacting the sucrose-6-acetate with the chlorinating reagent, to prepare a sucralose-6-acetate;
(c) reacting the sucralose-6-acetate with an acylating agent in the presence of a catalytic amount of I₂, to prepare 4,1′,6′-trichloro-4,1′,6′-trideoxy galactosucrose-penta-acetate; and
(d) deacetylating the 4,1′,6′-trichloro-4,1′,6′-trideoxy galactosucrose-penta-acetate, to prepare sucralose.

28. The process of claim 26, further comprising purifying the 4,1′,6′-trichloro-4,1′,6′-trideoxy galactosucrose-penta-acetate obtained in the reacting (c), prior to the deacetylating (d).

29. A process for preparing sucralose, comprising:
(a) crystallizing crude sucralose-6-ester in ethanol to obtain a first crystallized product;
(b) recrystallizing the first crystallized product in ethyl acetate to obtain purified sucralose-6-ester; and
(c) deesterifying the purified sucralose-6-ester, to prepare sucralose, or
(a′) crystallizing crude sucralose-6-ester in ethyl acetate to obtain a first crystallized product;
(b′) recrystallizing the first crystallized product in ethanol to obtain purified sucralose-6-ester; and
(c) deesterifying the purified sucralose-6-ester, to prepare sucralose.
